(19) United States
(12) Patent Application Publication
(10) Pub. No.: US 2008/0081064 A1
(43) Pub. Date: Apr. 3, 2008

(54) Implantable Medical Device with Apertures for Delivery of Bioactive Agents

(75) Inventors: Bruce M. Jelle, Chanhassen, MN (US); John V. Wall, Woodbury, MN (US); Aron B. Anderson, Minnetonka, MN (US)

Correspondence Address: KAGAN BINDER, PLLC, SUITE 200, MAPLE ISLAND BUILDING, 221 MAIN STREET NORTH, STILLWATER, MN, 55082, US

(73) Assignee: SurModics, Inc.

(21) Appl. No.: 11/904,925

(22) Filed: Sep. 28, 2007

Related U.S. Application Data
(60) Provisional application No. 60/847,824, filed on Sep. 28, 2006.

(51) Int. Cl. A61F 2/00; A61K 38/00; A61P 43/00

(52) U.S. Cl. 424/426; 514/2

(57) ABSTRACT

An implantable medical device for the release of bioactive agents at desired rates is described. The device includes a body member with two or more aperture sets and an inner space. The bioactive agents are arranged within the inner space of the body member so that each bioactive agent present in the inner space is individually releasable through their respective aperture set. The aperture sets modulate the release of the bioactive agents from the body member. Arrangement of apertures and bioactive agents within the inner portion chosen to provide desired and independent release rates from the device.

### CROSS-REFERENCE TO RELATED APPLICATIONS

This application claims the benefit of U.S. Provisional Patent Application Ser. No. 60/847,824, filed Sep. 28, 2006, entitled IMPLANTABLE MEDICAL DEVICE WITH APERTURES FOR DELIVERY OF BIOACTIVE AGENTS, the disclosure of which is incorporated herein by reference.

### FIELD OF THE INVENTION

The invention relates to implantable medical devices for delivering bioactive agents to a subject.

### BACKGROUND

Implantable medical devices having thin polymeric coatings containing therapeutic compounds that are released from the coating to provide a local therapeutic effect in the vicinity of the coated device have been shown to be valuable for the treatment of various medical conditions. For example, delivery of a therapeutic agent from the device surface can prevent cellular responses initiated by the presence of the implantable device. The therapeutic agent that is released from the coating can prevent conditions that would otherwise shorten the functional life of the device following implantation. The polymer system that forms the coating is able to regulate the release of the drug during a period of implantation in a patient. Such coated devices have been show to be particularly useful for the treatment of diseases of the cardiovascular system.

For example, stents having a polymeric coating containing a therapeutic agent can provide localized release of a therapeutic substance at the site of administration. Local administration of therapeutic agents via polymeric coatings on stents have shown favorable results in reducing restenosis. Several classes of drug-polymer chemistries have been explored for use in stent coatings as found in current art, some of which have been approved and are currently being used in medical procedures. Many of these chemistries are useful for delivering hydrophobic therapeutic agents.

While drug-eluting coatings have been shown to be very useful for these types of indications, this technology can have limitations. In many cases drug-eluting coatings are relatively thin. This can present restrictions on the upper limit of the amount of drug that can be loaded in the coating, and which can become available to a patient upon implantation of the device. Such restrictions can be problematic when the device is implanted in a patient for the long-term treatment of a medical condition, or if the treatment requires a substantial amount of drug to be delivered to a patient over a desired time course.

Another technical challenge in the technology of drug-delivering implantable devices relates to capability of delivering multiple drugs to a patient. This is not only challenging for devices where the drugs are to be delivered via a coating, but also more broadly to the area of other implantable drug delivery devices.

Since two or more drugs are to be delivered to a patient, this can present restrictions on the upper limit of the amount of drugs that can be present in the coating, and which can become available to a patient upon implantation of the device. For example, if two drugs are desired to be delivered from a particular device, this may reduce the amount of drug in the coating by about 50%. Such a significant reduction in the amount of drugs that are capable of being present in a coating may render the device useless.

Another challenge relates to delivering the drugs to a patient in a controlled manner. A particular polymer system may be useful for regulating the release of one class of drugs, but may not be capable of regulating the release of another class of drugs at a rate that is therapeutically useful to a patient. For example, if two drugs are capable of being present in a particular polymer coating, one of the drugs may be eluted in a desired profile, but the other drug may be eluted too quickly or may not be eluted at all.

The delivery of two or more drugs can even be more problematic if the drugs are incompatible in a particular polymer system intended to deliver the drugs. It is generally difficult, and usually impossible, to prepare a polymer composition that includes both a water soluble drug (such as a protein or polysaccharide) and a low molecular weight drug that is poorly soluble in water. In many cases, the polymer may be incompatible with solvent systems that are required to dissolve the hydrophilic drug. The hydrophilic drug may rapidly phase separate in an uncontrolled manner resulting in drug aggregation in the coating. This system can therefore produce coatings with unpredictable and variable release rate profiles. This situation is undesirable, as coatings displaying reproducible and controlled release rates cannot be formed.

### SUMMARY

In one aspect, the present invention provides implantable medical devices for the delivery of two or more bioactive agents to a subject. The invention also provides methods for the preparation of these implantable devices as well as methods for delivering two or more bioactive agents to a subject. The device provides desired release profiles for bioactive agents that, for example, have substantially different chemical properties and that may not be suitable for delivery in a mixture, or from a coating on a device.

In another aspect, the present invention provides implantable medical devices for the delivery of a bioactive agent wherein the bioactive agent is released at two individual rates from the device.

Generally, the device includes a body member with two or more aperture sets and an inner space. The bioactive agents are arranged within the inner space of the body member so that each bioactive agent present in the inner space is individually releasable through their respective aperture set. The aperture sets modulate the release of the bioactive agents from the body member. Arrangement of apertures and bioactive agents within the inner portion chosen to provide desired and independent release rates from the device.

In embodiments where a particular bioactive agent is released at two individual rates, bioactive agent present within the inner space is individually releasable through their respective aperture set. Therefore, in these embodiments the first bioactive agent and second bioactive agent can be the same.

The device of the present invention also provides advantages for treating a subject in that it allows the bioactive agents to be individually released from the device in a desired manner. Upon implantation in a subject, two or more bioactive agent release profiles can be achieved. The release profiles can be independent of one another, meaning that the release of one bioactive agent does not undesirably impact the release of one (or more) of the other bioactive agents.

Beneficially, for some aspects of the present invention, the device can be used to deliver two or more bioactive agents that are mutually incompatible. Since the bioactive agents are at least predominantly physically separated in the inner space, and releasable through their respective aperture sets, mixing of the bioactive agents is not required. For example, the device can be used to deliver bioactive agents having different solubility characteristics.

From this standpoint, the device of the present invention is particularly advantageous as two types of bioactive agents having different solubility characteristics can be delivered from the same device, both bioactive agents being released in a desired manner, and, if desired, in the same therapeutic window. This arrangement can provide many benefits to a patient, particularly when the presence of the two bioactive agents results in a therapeutic advantage over administration of one bioactive agent.

Administration of two or more bioactive agents can be therapeutically advantageous for overcoming disease resistance. As an example, a first bioactive can be released from the device in a short term burst to treat a condition associated with a bacterial or viral infection in the body, and a second bioactive agent can be released from the device over a longer period of time to prevent the emergence of a pathogen that is resistant to the first bioactive agent.

The body member provides other distinct benefits for release of the bioactive agents. For example, the body member provides increased loading of the bioactive agents in the device.

In one aspect, the invention provides an implantable device for the delivery of at least two bioactive agents to a subject. The device includes a body member comprising a first set of apertures, a second set of apertures, and an inner space. The inner space comprises an amount of a first bioactive agent and an amount of a second bioactive agent. During implantation in a subject, the majority of the amount of the first bioactive agent is releasable from the device through the first set of apertures, and the majority of the amount of the second bioactive agent is releasable from the device through the second set of apertures.

In certain embodiments the inner space comprises at least one polymeric matrix, which the first and second bioactive agents are present in and releasable from. A wide variety of polymeric delivery matrices may be used with the body member. The body member can provide integrity to the drug within the inner portion and can provide integrity to a polymeric matrix. The body member can also provide protection to the polymeric matrix to prevent the polymeric matrix from being abraded if the device is moved within the body. If desired, the device of the invention allow use of polymer systems that are desirable for bioactive agent release but that may not form good coatings.

In more specific embodiments, the inner space can include two different polymeric matrices (e.g., first and second polymeric matrices), wherein the first and second bioactive agents are individually disposed in these matrices. The first polymeric matrix can include a hydrophobic bioactive agent and the second polymeric matrix can include a hydrophilic bioactive agent.

In some aspects the device includes a bioactive agent comprising a polypeptide. Examples of polypeptides that can be delivered from the device include peptides, enzymes, enzyme inhibitors, hormone polypeptides, growth factors, cytokines, lymphokines, matrix proteins, serum proteins, antibodies, antibody fragments, and peptide antigens. In some desired aspects, the polypeptide is present in a biodegradable polymeric matrix. Upon degradation of the matrix, the polypeptide can be released from the device through the apertures.

In another aspect, the device includes a body member comprising a first set of apertures, a second set of apertures, and an inner space. The inner space comprises a first bioactive agent and a second bioactive agent, wherein the first bioactive agent and the second bioactive agent are substantially unmixed in the inner space. During implantation in a subject, the first bioactive agent is releasable from the device through the first set of apertures, and the second bioactive agent is releasable from the device through the second set of apertures.

The invention also provides a method for forming an implantable bioactive agent delivery device. The method includes a step of obtaining a body member comprising a first set of apertures, a second set of apertures, and an inner space. The method also includes a step of providing a first bioactive agent to a portion of the inner space so the first bioactive agent is primarily releasable through the first set of apertures during implantation of the device in a subject. The method also includes a step of providing a second bioactive agent to a portion of the inner space so the second bioactive agent is primarily releasable through the second set of apertures during implantation of the device in a subject.

Providing two or more bioactive agents in the inner area circumvents the need for elaborate body member constructions (such as multiple lumen arrangements). The inner space also provides a way for increasing the amount of bioactive agent(s) that can be loaded into the device. Given the teaching herein, the drug delivery device is also easy to fabricate.

The invention also provides a method for delivering two or more bioactive agents to a subject. The method includes a step of implanting at a target location in the body an implantable bioactive agent delivery device. The device comprises a body member comprising a first set of apertures, a second set of apertures; and an inner space. The inner space includes a first bioactive agent, and a second bioactive agent; wherein the first bioactive agent and the second bioactive agent are substantially unmixed in the inner space. The method also includes a step of allowing release of the first bioactive agent through the first set of apertures and release of the second bioactive agent through the second set of apertures in the body.

### BRIEF DESCRIPTION OF THE DRAWINGS

FIG. 1A is a schematic illustration of one embodiment of the delivery device having a cylindrical shape, an aperture set with large apertures, and an aperture set with small apertures.

FIG. 1B is a cross sectional view of the delivery device of FIG. 1A showing the arrangement of bioactive agents within the inner space.

FIG. 2 is a schematic illustration of another embodiment of the delivery device having a cylindrical shape an aperture set with large apertures and an aperture set with small apertures.

FIG. 3 is a cross sectional view of another embodiment of the delivery device showing the arrangement of bioactive agent within the inner space.

FIG. 4 is a cross sectional view of another embodiment of the delivery device showing the arrangement of bioactive agent within the inner space.

FIG. 5 is a cross sectional view of another embodiment of the delivery device showing the arrangement of bioactive agent within the inner space.

FIGS. 6A-6D are cross sectional view of a portion of a delivery device with a biodegradable matrix having bioactive agent, and showing the degradation of the matrix at different time periods.

### DETAILED DESCRIPTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

Generally, the present invention is directed towards methods and devices for the delivery of two or more bioactive agents to a subject. The bioactive agents are delivered to the subject after implantation of the delivery device at a target location in the body. As used herein, “implantation” refers to a process wherein the delivery device is placed at a target location in the body, wherein the delivery device is designed to reside at the target location for a period of time. During that period of time all or a portion of the bioactive agents are released from the delivery device and provide a therapeutic effect to the subject. All, or portions of the amounts of bioactive agents can be delivered from the device during the implantation period. The device can be configured to individually deliver the bioactive agents at selected rates during implantation.

The present invention describes various embodiments of delivery devices for bioactive agents, methods for preparing these devices, and methods for delivering two or more bioactive agents to a subject using these devices. Basic features define the devices and methods of the invention. The invention also describes a number of more specific features of these devices and methods. The disclosure of the present invention also relates to these more specific features that can be used with the basic features of the invention. The invention encompasses combinations of features that are more specifically described in the application. For purposes of brevity, these more specific features are often described individually in the application; however, it is understood that the present disclosure supports combinations of specific features with the basic features of the invention.

The body member of the delivery device is generally a receptacle structure that houses the bioactive agents. The body member can be of any suitable size and/or shape for implantation into a target area of the body. Generally, the target area within a subject will have known anatomical features, and the body member can be designed to reside at the target location based on these anatomical features. The basic features of the body member include a wall, apertures within the wall through which the bioactive agents are released, and an inner space that houses the bioactive agents.

Generally, the body member is constructed so that release of the bioactive agents predominantly or entirely occurs through the apertures in the body member. The body member may partially or completely prevent passage of the bioactive agents through the material of the body member. In other words, the walls of the body member may have limited or no permeability to the bioactive agents in the inner space of the delivery device.

The body member can be constructed of a synthetic or natural material that is suitable for use within the body. These materials are typically compatible with body fluids and/or tissues and do not elicit an adverse response when the device is implanted in a subject.

In some cases, the body member is formed of one or more metals or metal alloys. Examples of suitable metals include platinum, gold, or tungsten, as well as other metals such as rhenium, palladium, rhodium, ruthenium, titanium, nickel, and alloys of these metals, such as stainless steel, titanium/nickel, nitinol alloys, cobalt chrome alloys, non-ferrous alloys, and platinum/iridium alloys. One exemplary alloy is MP35. Any suitable .metal, including other alloys or combinations, can be used to form the delivery device.

The delivery device can also be formed from a plastic polymer. Plastic polymers include those formed of synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, vinylidene difluoride, and styrene. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polydimethylsiloxanes, and polyetherketone.

Other suitable polymers that can be used to construct the body member include polyamides, polyimides, polyolefins, polystyrenes, polyesters, polycarbonates, polyketones, polyureas, acrylonitrile butadiene, butadiene rubber, chlorinated and chlorosulfonated polyethylene, chloroprene, ethylene-propylene rubber (EPM, EPDM), polyethylene (PE)-EPDM, polypropylene (PP)-EPDM, ethylene vinyl alcohol (EVOH), epichlorohydrin, isobutylene isoprene, isoprene, polysulfides, silicones, nitrile/PVC resin blends (NBR/PVC), styrene butadienes, and vinyl acetate ethylenes, and combinations thereof.

In some aspects all or a portion of the body member is formed of a biodegradable material. If a biodegradable material is used, generally the body member will degrade at a rate that is slower than the release of the bioactive agents from the inner portion of the body member. In this regard, the body member will still be able to modulate the release of the bioactive agents during the release period. After most or all of the amounts of bioactive agents are released from the device, the body member erodes. The degradation products may be used by and/or excreted from the body.

A body member that is degradable within the body can be fabricated from natural or synthetic polymeric materials. Synthetic polymeric materials that are well known in the art and that can be used to prepare a biodegradable body member include polyanhydrides, polycaprolactone, polyglycolic acid, poly-L-lactic acid, poly-D-L-lactic, poly(D-lactic-co-glycolic acid), poly(D,L-lactic-co-glycolic acid), poly(ε-caprolactone), poly(lactic acid-co-lysine), poly(lactic acid-co-trimethylene carbonate), poly(valerolactone), poly(hydroxy butyrate), poly(hydrovalerate), polyphosphate esters, poly(hydroxybutyrate), polycarbonate, polyanhydride, poly(ortho esters), poly(phosphoesters), polyesters, polyamides, polyphosphazenes, poly(p-dioxane), poly(amino acid), polydioxanone, poly(propylene fumarate), poly(ethyleneoxide), and poly(butyleneterephthalate).

In some case, in order to provide a body member with a rate of degradation that is slower than the rate of release of the bioactive agent, synthetic biodegradable polymers such as poly-L-lactic acid, poly-D-L-lactic acid, and poly(ε-caprolactone), which are a relatively slow-bioabsorbing material (months to years), can be used as the primary degradable component of the body member.

The biodegradable body member can also be formed using natural biodegradable polysaccharides. Natural biodegradable polysaccharides having pendent coupling groups, such as polymerizable groups, can be reacted to form a body member with a cross-linked matrix of polysaccharides. Desirably, the natural biodegradable polysaccharides are low molecular weight polymers, such as having a molecular weight of about 50,000 Da or less, 25,000 Da or less, or 10,000 Da or less.

Natural biodegradable polysaccharides with pendent coupling groups are described in U.S. Pub. No. 2005/0255142, published Nov. 17, 2005, (Chudzik et al) and U.S. patent application Ser. No. 11/271,213, filed Nov. 11, 2005 (Chudzik et al.), both commonly assigned to the applicant of the present invention. One preferred class of natural biodegradable polysaccharides are selected from the group of maltodextrin, amylose, and polyalditol.

A body member having a rate of degradation that is slower than the rate of release of the bioactive agents can be formed by using polysaccharides that are highly derivatized with pendent coupling groups. For example, the derivatization level can be above 0.3 μmoles/mg, and preferably around about 0.7 μmoles/mg. Use of a highly derivatized polysaccharide can result in the formation of a body member with a tightly formed polymeric matrix that has increased resistance to degradation.

The body member can also be formed using polysaccharides derivatized with hydrophobic moieties. This can decrease the water solubility of the polysaccharides and make a body member more hydrophobic and resistant to degradation when the device is placed in the body. Exemplary hydrophobic polysaccharides can be prepared according to methods described in U.S. Patent Application No. 60/782,957 (Chudzik, S. J.), filed Mar. 15, 2006, and assigned to the applicant of the present invention. The body member can be formed using a hydrophobic moiety derivatized with hydrophobic moieties comprising a C₂-C₁₈, linear, branched, or cyclic alkyl group, or a C₂-C₁₀, or a C₂-C₆, linear, branched, or cyclic alkyl group. In some aspects, the hydrophobic derivative of a natural biodegradable polysaccharide has a degree of substitution of greater than 1.

Other materials that can be used to form the body member are those that include human tissue such as bone, cartilage, skin and teeth; or other organic materials such as wood, cellulose, compressed carbon, and rubber. Other contemplated biomaterials include ceramics including, but not limited to, silicon nitride, silicon carbide, zirconia, and alumina, as well as glass, silica, and sapphire.

If desired, the body member can be constructed to have a degree of flexibility. If the device is flexible, force can be applied to one or more portions of the device to change its configuration, and when the force is released, it reverts back to its original configuration.

The body member can also be constructed to have a degree of bendability. If the device is bendable, force can be applied to one or more portions of the device to change its configuration, and that configuration is maintained when the force is released. If the device has a degree of flexibility and/or bendability, it can minimize disruption of the tissue that it is being inserted into.

In either case, if the device is constructed to be flexible or bendable, any force that is applied to the body member to change its configuration does not cause fracturing of the body member. Devices that are bendable or flexible can facilitate the process of insertion of the device at the target location in a subject. For example, the device can be inserted into the target portion of the body using an instrument such as a catheter or other insertion tool. During the insertion process it may be necessary to flex or bend the device to properly deliver or place the device at the target site. For example, delivery of the device through the vasculature via a catheter often involves navigation through a tortuous pathway for placement at a target site. The delivery device may be flexed at one or more points during the insertion process. Various thermoplastic, thermoset, and metal materials can be used to construct a flexible delivery device.

The wall of the body member generally has a thickness that will provide adequate structural support to the device. With this in mind, the body member can include a thinner wall if stronger structural materials, such as metals, are used. In some aspects, the wall has a thickness of about 1000 μm or less. In some aspects, the wall has a thickness in the range of about 100 μm to about 400 μm. The thickness of the wall can be uniform over the entire body member, or can be different at more than one locations of the body member.

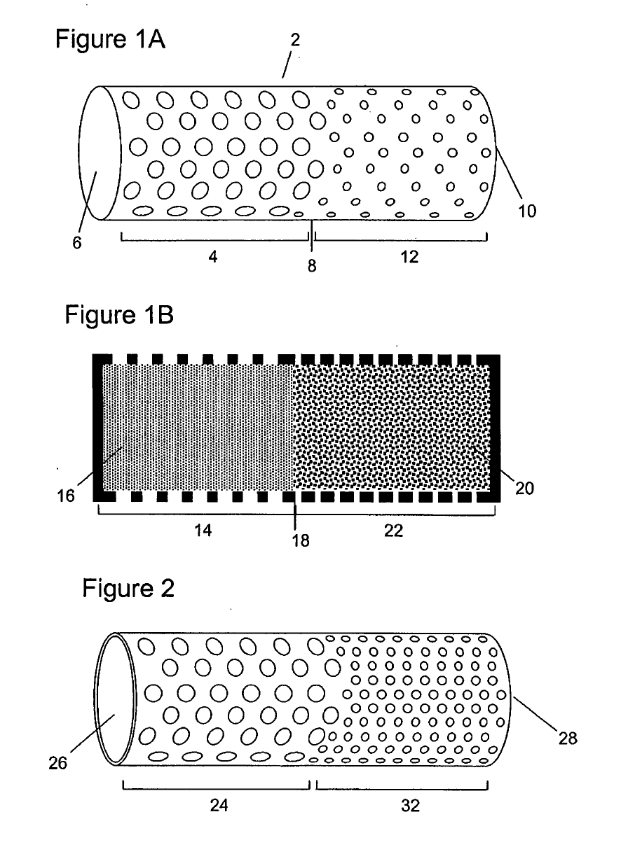

The body member can have any desired shape. In some aspects, the body member has an elongated shape, as exemplified by tubular and cylindrical shapes. FIG. 1A shows a body member having a cylindrical shape.

A cross section of the body member (as viewed from the end of the delivery device) shows that the body member includes a rounded shape. The body member can also have other cross-sectional shapes, which can provide the body member with linear or rounded surfaces. Other rounded shapes include an oval shape. The cross-sectional shape can also include a straight portion. For example, the cross section can have a polygonal shape, such as triangular, square, rectangular, hexagonal, octagonal, etc.

The body member can also have a flattened shape, exemplified by pillow shapes. The body member can also have a spherical shape. The body member can also have an irregular shape, and can include combinations of linear, rounded, convex, or concave surfaces.

The device can also have particular configurations. For example, the cylindrically shaped body member can be constructed in a non-linear configuration. The body member may still have a cross-sectional shape that is rounded (such as a circular cross sectional shape). However, the axis of the body member, running from a proximal end to a distal end would follow a non-linear path. For example, the axis of the body member can be curved at one or more points.

A device with a non-linear shaped body can greatly increase the surface from which the bioactive agents are released. In addition, increased amounts of bioactive agents can be loaded in the body member. Given this, a non-linear configuration in combination with the arrangement of apertures and bioactive agents provides a particularly useful way of delivering relatively substantial amounts of two or more bioactive agents to a subject.

A body member having a non-linear curved shape can include coiled and helical shapes. The body member may have a non-linear shape as described in U.S. Pat. No. 6,719,750 B2 (“Devices for Intraocular Drug Delivery,” Varner et al.).

The device can be of a size suitable for implantation into a desired area of the body. Generally, the device is small, such that it can be implanted in a subject without the significant disruption of tissue during the implantation process. The delivery device can be measured in various ways, such as by the length, width, or height of the device, or combinations thereof The device can also be measured by its displacement (i.e., the volume that it occupies).

For example, referring to FIG. 1A, the body member has a length (L) along the axis of the body member from the first end the second end. The body member also has a diameter (D).

In some aspects, the device has a length (such as along the length of the axis of the device) of about 4 cm or less. One particularly useful range of lengths is in the range of about 5 mm to about 4 cm.

In some aspects, the body member has a width (such as along the diameter of a cross section of the body member) of about 2 mm or less. One particularly useful range of widths is from about 0.5 mm to about 2 mm.

The body member can also be measured by its cross sectional area. In some aspects, the body member has a cross sectional area of about 3.2 mm² or less. One particularly useful range of cross sectional areas is from about 0.2 mm² to about 3.2 mm².

In configurations wherein the body member has a non-linear shape, the overall length of the body member (following the non-linear path) may be greater than the length of the device. For example, for delivery devices having a helically-shaped body member, the length of the body member (as measured along its helical path) can be up to about 4 cm. The overall length of the device can be about 1 cm or less. The overall width of the device can be about 0.5 cm or less.

The body member can also have one or more optional feature(s) that can be used to facilitate the insertion and/or removal of the delivery device into and/or from a subject. For example, the delivery device can include a portion that can be affixed to an insertion/retraction instrument. Such an optional feature is exemplified by a cap structure present on the proximal end of a drug delivery device as described in U.S. Pat. No. 6,719,750 B2 (“Devices for Intraocular Drug Delivery,” Varner et al.). For insertion into a target area of the eye, the cap is engaged by an insertion instrument, which can be used to rotatably advance the device into the eye. An exemplary insertion tool for an ocular bioactive agent delivery device is described in pending U.S. application Ser. No. 11/436,277 (Varner et al.), filed May 18, 2006, and assigned to the applicant of the present invention.

The device includes aperture sets from which the bioactive agents present within the inner space of the body member are released. An aperture “set” can include one aperture or more than one aperture. In many aspects of the invention an aperture set includes a plurality of apertures. While there is no particular upper range for the number of apertures that can be present in an aperture set, the number of apertures may be dictated by various aspects of the device. These aspects include one or more of the surface area of the body member of the device, the arrangement of the bioactive agent within the body member, the amount of bioactive agents within the body member, and the desired delivery rate of the bioactive agents from the device.

For example an aperture set can include one, two, three, four, five, six, seven, eight, nine, ten, or more apertures per set. In some aspects, the first and second aperture sets have a number of apertures in the range of about 10 to about 100 apertures, and in some aspects in the range of about 20 to about 50 apertures.

In many aspects, the aperture sets are grouped. A grouped set of apertures refers to apertures of a set that are physically adjacent to one another. For example, a grouped set of apertures within a set can be in a cluster. In a cluster the apertures are not dispersed by apertures of another group. If the aperture sets include sets of grouped apertures, the sets can be segregated on the body member in any desired arrangement.

In some aspects the aperture sets are segregated on the body member along the length of the device. Reference is made to FIGS. 1A and 1B. These figures show a first set of apertures 4 formed in an area of the body member 2 from the first end 6 to point 8. A second set of apertures 12 is formed in an area of the body member 2 from point 8 to the second end 10.

As shown in FIG. 1B, which illustrates a cross sectional view of the device of FIG. 1A, the first bioactive agent 16 is shown adjacent to and releasable through the first aperture set 14. Accordingly, the second bioactive agent 20 is adjacent to and releasable through the second aperture set 22.

In FIGS. 1A and 1B, the apertures of the first set are shown as having a larger average aperture size than the apertures of the second aperture set, although the number of apertures in the first and second sets are approximately the same. This decreases the total number of apertures of the second set, and is one way of reducing the rate of release of the second bioactive agent from the device.

While FIGS. 1A and 1B illustrate that the ends of the cylindrical device are closed (i.e., the body member seals off the ends of the cylinder), one or both ends of the device can be open. These one or more openings can represent one or more additional apertures in the body member. If these one or more apertures are present, bioactive agent can also be released through these aperture(s).

FIG. 2 shows a device with additional apertures at the ends of the device. The device includes a first aperture set 24 with a plurality of small apertures and one large aperture 26 the first end, and a second aperture set 32 with a plurality of very small apertures and one large aperture 28 at the second end.

The first and second aperture sets can include the same number of apertures, or a different number of apertures. If more than two aperture sets are present in the device, the sets can include the same number of apertures, or a different number of apertures. FIG. 2 shows a device with having a greater number of apertures in the second set 32 than the first set 24. However, if the average size of the apertures is greater in the first set, then the difference in the total aperture area between the first and second aperture sets may be small, or may be the same.

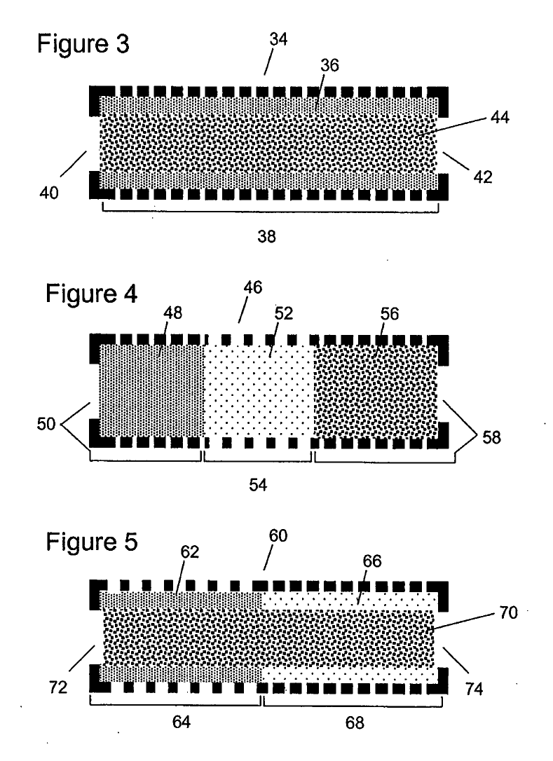

FIG. 3 shows another embodiment of the present invention. In this embodiment, the bioactive agents are radially segregated in the body member 34. In the device, the first bioactive agent 36 is adjacent to the first aperture set 38. For example, the first bioactive agent 36 can be present in a cylindrically-shaped polymer matrix in contact with the inner surface of the wall of the body member 34. The first aperture set 38 includes a plurality of smaller apertures on the circumference of the body member 34. The second aperture set that has two large apertures represented by the openings at the first 40 and second ends 42 of the device. Release of the first bioactive agent 36 occurs predominantly through the first aperture set 38 and release of the second bioactive agent 44 occurs predominantly through the second aperture set (openings 40 and 42).

FIG. 4 shows another embodiment of the present invention. In this embodiment, the bioactive agents are segregated along the axis of the body member. The delivery device includes three bioactive agents in the body member 46. The bioactive agents are segregated along the (lengthwise) axis of the device. The first bioactive agent 48 is releasable from a first set 50 of apertures, which includes a large aperture on the first end of the body member and a plurality of smaller apertures disposed on the circumferential face of the body member near the first end. The second bioactive agent 52 is releasable from a second set 54 of apertures which includes a plurality of smaller apertures disposed on the circumferential face of the body member between the first and second ends. The third bioactive agent 56 is releasable from a third set of apertures 58, which includes a large aperture on the second end of the device and a plurality of smaller apertures disposed on the circumferential face of the body member near the second end.

FIG. 5 shows another embodiment of the present invention. In this embodiment, the bioactive agents are segregated both radially and along the axis of the body member. The delivery device includes three bioactive agents in the body member 60. The first bioactive agent 62 is releasable from a first set of apertures 64, which includes a plurality of smaller apertures disposed on the circumferential face of the body member near the first end. The second bioactive agent 66 is releasable from a second set of apertures 68, which includes a plurality of smaller apertures disposed on the circumferential face of the body member near the second end. The third bioactive agent 70 is releasable from a third set of apertures including two large apertures (72 and 74) represented by the openings at the first and second ends of the device.

The device can include additional aperture sets, such as a fourth, fifth, sixth, etc. aperture set. A device having a plurality of aperture sets can be segregated in any desired manner in the body member, such as along any axis of the body member (e.g., lengthwise) or radially within the body member. In this manner, the bioactive agents can be present in the inner space of the body member in “zones.”

The aperture sets of the body member have openings of a size that can regulate the release of the bioactive agents from the inner space of the device. A particular aperture set can have a total aperture area, referring to the total area of the apertures in that set. Generally the rate of release of a bioactive agent from an aperture set can be reduced by reducing the total area in that aperture set, and the rate of release can be increased by increasing the total area.

Given the size of the device, the apertures of the device generally have a small (open) area. For example, an individual aperture can have an area of about 12.5 mm² or less. In some aspects, an individual aperture can have an area in the range of about range of 3×10³ μm² to 3.25×10⁵ μm².

The device can also be defined in terms of the relationship between the surface area of the body member and the total aperture area. Generally the rate of release of a bioactive agent from an aperture set can be reduced by reducing the ratio of the total aperture area to the total body member area. In some aspects of the invention, the ratio of the total aperture area to the total body member area is 2:3 or less. In more specific aspects, the ratio of the total aperture area to the total body member area is in the range of 1:10 to 1:207.

An aperture can have any shape, such as a circular, oval shape, triangular, square, or rectangular shape. The body member can be fabricated with combinations of shapes of apertures.

The device of can also be prepared so that one or more apertures (for example, one set of apertures, or more than one set of apertures) include a material that is dissolvable or degradable during implantation in a subject. For example, the apertures can be plugged or covered with a biodegradable polymeric material, such as one described herein. This can delay or modulate release of bioactive agent from the device.

The invention provides an ideal device for the controlled release of two or more bioactive agents. Two or more bioactive agents are disposed within the body member in such a manner that they are released through their respective aperture sets when the device is placed within the subject. Generally, the device is formed by disposing the bioactive agents within the inner space of the body member so that the bioactive agents are at least predominantly separated in the inner space.

In order to provide a biodegradable system, the bioactive agents are included in the degradable body member. The bioactive agents can be provided alone, with other inert ingredients that are released from the device, or within one or more biodegradable matrices disposed within the body member.

The term “bioactive agent,” refers to an inorganic or organic molecule, which can be synthetic or natural, that causes a biological effect when administered in vivo to an animal, including but not limited to birds and mammals, including humans.

Two or more bioactive agents can be present in the bioactive space and releasable through the apertures. Given the ease of fabrication, in some cases the device can be prepared to deliver, three, four, five, or more bioactive agents from the body member.

In some aspects of the invention, the device is used to deliver bioactive agents having different physical properties. The different physical properties may otherwise cause the bioactive agents to be uncombinable, undeliverable if mixed together, or releasable from the device in a manner that is not desired.

In some aspects the delivery device includes bioactive agents having different solubilities in a selected liquid. Solubility refers to the level to which a solute dissolves in a solvent. For a bioactive agent in a particular solvent, “practically insoluble”, or “insoluble” refers to having a solubility of 1 part agent per more than 10,000 parts of solvent, “very slightly soluble” refers to having a solubility of from 1 part agent per 1000 to 10,000 parts of solvent; “slightly soluble” refers to having a solubility of 1 part agent per from 100 to 1000 parts of solvent; “sparingly soluble” refers to having a solubility of 1 part agent from 30 to 100 parts of solvent; “soluble” refers to having a solubility of at least 1 part agent per from 10 to 30 parts solvent, “freely soluble” refers to having a solubility of at least 1 part agent per from 1 to 10 parts solvent, or “very soluble” refers to having a solubility of greater than 1 part agent per from 1 part solvent. These descriptive terms for solubility are standard terms used in the art (see, for example, Remington: The Science and Practice of Pharmacy, 20^{th} ed. (2000), Lippincott Williams & Wilkins, Baltimore Md.).

For example, the device can include a first bioactive agent that is soluble in an organic solvent such as tetrahydrofuran (THF), and a second bioactive agent that is poorly or insoluble in the organic solvent. One example of such a combination would be a compound such as rapamycin, and a water or methanol soluble compound such as vincristine sulfate. As another example, the device can include a first bioactive agent that is soluble in a polar liquid such as water, and a second bioactive agent that is poorly or insoluble in the polar liquid. One example of such a combination would be a protein such as IgG, and a steroid compound such as triamcinolone acetonide

In some aspects, the device of the invention provides release of a hydrophilic bioactive agent and a hydrophobic bioactive agent. The hydrophilic and hydrophobic bioactive agents can be released at a rate to provide a therapeutic effect within the same window.

In other aspects the delivery device includes bioactive agents having different molecular weights. The device can be used to control the release of these bioactive agents in a desired manner if the size of the bioactive agents causes substantial differences in the release rates of the bioactive agents. In some aspects the first bioactive agent is a low molecular weight compound and the second bioactive agent is a high molecular weight compound. For example, in some aspects the molecular weight of the first bioactive agent is less than 1000 Da and the weight of the second bioactive agent is greater than 1000 Da. Examples of low and high molecular weight bioactive agent combinations include a first bioactive agent selected from the groups consisting of antiproliferatives and the second bioactive agent is selected from the group consisting of proteins, oligo and polynucleotides, and polysaccharides.

The bioactive agents can be present in the delivery device in amount to provide a desired therapeutic response during a period of time the device is implanted in a subject. The amounts of bioactive agents placed within the delivery device may depend on one or more factors including: the potency of the bioactive agents, the in vivo lifetime of the bioactive agents, and the desired release rate of the drugs. While the invention is not limited to any particular amount of bioactive agents that are present within the device, amounts of bioactive agents present can be in very small amounts, such as in picogram amounts, up to very large quantities, such as gram amounts. Bioactive agents such as 5-fluorouracil or 5-fluorouridine have been shown to provide therapeutic effects in very small amounts. Other bioactive agents such as paclitaxel or estradiol can be delivered to a subject in relatively substantial quantities. Exemplary ranges of bioactive agents are from about 100 pg to about 10 grams, from about 10 ng to about 500 mg, and from about 1 μg to about 10 mg, and from about 10 μg to about 1 mg.

While the device is advantageous for the delivery of two or more bioactive agents that are different, the device can also be prepared to deliver the same bioactive agent from the two or more aperture sets in the device. For example, in some cases it may be desirable to deliver the first bioactive agent very rapidly from the device and also deliver the same bioactive agent from the device over a longer period of time. In other words, the device can provide a short term burst and the long term release of a particular bioactive agent, as modulated by the properties of the delivery device.

A partial list of bioactive agents is provided below. One may choose any one of the hydrophilic bioactive agents to be included in a microparticle set alone, or in combination with any other bioactive agent. A comprehensive listing of bioactive agents, in addition to information of the water solubility of the bioactive agents, can be found in The Merck Index, Thirteenth Edition, Merck & Co. (2001).

The matrices prepared according to the invention can be used to release bioactive agents falling within one or more of the following classes include, but are not limited to: ACE inhibitors, actin inhibitors, analgesics, anesthetics, anti-hypertensives, anti polymerases, antisecretory agents, anti-AIDS substances, antibiotics, anti-cancer substances, anti-cholinergics, anti-coagulants, anti-convulsants, anti-depressants, anti-emetics, antifungals, anti-glaucoma solutes, antihistamines, antihypertensive agents, anti-inflammatory agents (such as NSAIDs), anti metabolites, antimitotics, antioxidizing agents, anti-parasite and/or anti-Parkinson substances, antiproliferatives (including antiangiogenesis agents), anti-protozoal solutes, anti-psychotic substances, anti-pyretics, antiseptics, anti-spasmodics, antiviral agents, calcium channel blockers, cell response modifiers, chelators, chemotherapeutic agents, dopamine agonists, extracellular matrix components, fibrinolytic agents, free radical scavengers, growth hormone antagonists, hypnotics, immunosuppressive agents, immunotoxins, inhibitors of surface glycoprotein receptors, microtubule inhibitors, miotics, muscle contractants, muscle relaxants, neurotoxins, neurotransmitters, polynucleotides and derivatives thereof, opioids, photodynamic therapy agents, prostaglandins, remodeling inhibitors, statins, steroids, thrombolytic agents, tranquilizers, vasodilators, and vasospasm inhibitors.

Antibiotics are recognized and are substances which inhibit the growth of or kill microorganisms. Examples of antibiotics include penicillin, tetracycline, chloramphenicol, minocycline, doxycycline, vancomycin, bacitracin, kanamycin, neomycin, gentamycin, erythromycin, cephalosporins, geldanamycin, and analogs thereof Examples of cephalosporins include cephalothin, cephapirin, cefazolin, cephalexin, cephradine, cefadroxil, cefamandole, cefoxitin, cefaclor, cefuroxime, cefonicid, ceforanide, cefotaxime, moxalactam, ceftizoxime, ceftriaxone, and cefoperazone.

Antiseptics are recognized as substances that prevent or arrest the growth or action of microorganisms, generally in a nonspecific fashion, e.g., by inhibiting their activity or destroying them. Examples of antiseptics include silver sulfadiazine, chlorhexidine, glutaraldehyde, peracetic acid, sodium hypochlorite, phenols, phenolic compounds, iodophor compounds, quaternary ammonium compounds, and chlorine compounds.

Anti-viral agents are substances capable of destroying or suppressing the replication of viruses. Examples of anti-viral agents include α-methyl-P-adamantane methylamine, hydroxy-ethoxymethylguanine, adamantanamine, 5-iodo-2′-deoxyuridine, trifluorothymidine, interferon, and adenine arabinoside.

Enzyme inhibitors are substances that inhibit an enzymatic reaction. Examples of enzyme inhibitors include edrophonium chloride, N-methylphysostigmine, neostigmine bromide, physostigmine sulfate, tacrine HCl, tacrine, 1-hydroxymaleate, iodotubercidin, p-bromotetramisole, 10-(α-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N-monomethyl-L-arginine acetate, carbidopa, 3-hydroxybenzylhydrazine HCl, hydralazine HCl, clorgyline HCl, deprenyl HCl, L(−), deprenyl HCl, D(+), hydroxylamine HCl, iproniazid phosphate, 6-MeO-tetrahydro-9H-pyrido-indole, nialamide, pargyline HCl, quinacrine HCl, semicarbazide HCl, tranylcypromine HCl, N,N-diethylaminoethyl-2,2-diphenylvalerate hydrochloride, 3-isobutyl-1-methylxanthine, papaverine HCl, indomethacin, 2-cyclooctyl-2-hydroxyethylamine hydrochloride, 2,3-dichloro-α-methylbenzylamine (DCMB), 8,9-dichloro-2,3,4,5-tetrahydro-1H-2-benzazepine hydrochloride, p-aminoglutethimide, p-aminoglutethimide tartrate, R(+), p-aminoglutethimide tartrate, S(−), 3-iodotyrosine, alpha-methyltyrosine, L(−) alpha-methyltyrosine, DL(−), cetazolamide, dichlorphenamide, 6-hydroxy-2-benzothiazolesulfonamide, and allopurinol.

Anti-pyretics are substances capable of relieving or reducing fever. Anti-inflammatory agents are substances capable of counteracting or suppressing inflammation. Examples of such agents include aspirin (salicylic acid), indomethacin, sodium indomethacin trihydrate, salicylamide, naproxen, colchicine, fenoprofen, sulindac, diflunisal, diclofenac, indoprofen and sodium salicylamide. Local anesthetics are substances that have an anesthetic effect in a localized region. Examples of such anesthetics include procaine, lidocaine, tetracaine and dibucaine.

Cell response modifiers are chemotactic factors such as platelet-derived growth factor (pDGF). Other chemotactic factors include neutrophil-activating protein, mono cyte chemoattractant protein, macrophage-inflammatory protein, SIS (small inducible secreted) proteins, platelet factor, platelet basic protein, melanoma growth stimulating activity, epidermal growth factor, transforming growth factor (alpha), fibroblast growth factor, platelet-derived endothelial cell growth factor, insulin-like growth factor, nerve growth factor, and bone growth/cartilage-inducing factor (alpha and beta). Other cell response modifiers are the interleukins, interleukin inhibitors or interleukin receptors, including interleukin 1 through interleukin 10; interferons, including alpha, beta and gamma; hematopoietic factors, including erythropoietin, granulocyte colony stimulating factor, macrophage colony stimulating factor and granulocyte-macrophage colony stimulating factor, tumor necrosis factors, including alpha and beta; transforming growth factors (beta), including beta-1, beta-2, beta-3, inhibin, activin, and DNA that encodes for the production of any of these proteins.

Examples of statins include lovastatin, pravastatin, simvastatin, fluvastatin, atorvastatin, cerivastatin, rosuvastatin, and superstatin.

Examples of steroids include glucocorticoids such as cortisone, hydrocortisone, dexamethasone, betamethasone, prednisone, prednisolone, methylprednisolone, triamcinolone, beclomethasone, fludrocortisone, and aldosterone; sex steroids such as testostersone, dihydrotestosterone, estradiol, diethylstilbestrol, progesterone, and progestins.

Exemplary ligands or receptors include antibodies, antigens, avidin, streptavidin, biotin, heparin, type IV collagen, protein A, and protein G.

Exemplary antibiotics include antibiotic peptides.

The bioactive agent can provide antirestenotic effects, such as antiproliferative, anti-platelet, and/or antithrombotic effects. In some embodiments, the bioactive agent can include anti-inflammatory agents, immunosuppressive agents, cell attachment factors, receptors, ligands, growth factors, antibiotics, enzymes, nucleic acids, and the like. Compounds having antiproliferative effects include, for example, actinomycin D, angiopeptin, c-myc antisense, paclitaxel, taxane, and the like.

Representative examples of bioactive agents having antithrombotic effects include heparin, heparin derivatives, sodium heparin, low molecular weight heparin, hirudin, lysine, prostaglandins, argatroban, forskolin, vapiprost, prostacyclin and prostacyclin analogs, D-ph-pr-arg-chloromethylketone (synthetic antithrombin), dipyridamole, glycoprotein IIb/IIIa platelet membrane receptor antibody, coprotein IIb/IIIa platelet membrane receptor antibody, recombinant hirudin, thrombin inhibitor (such as commercially available from Biogen), chondroitin sulfate, modified dextran, albumin, streptokinase, tissue plasminogen activator (TPA), urokinase, nitric oxide inhibitors, and the like.

The bioactive agent can also be an inhibitor of the GPIIb-IIIa platelet receptor complex, which mediates platelet aggregation. GPIIb/IIIa inhibitors can include monoclonal antibody Fab fragment c7E3, also know as abciximab (ReoPro™), and synthetic peptides or peptidomimetics such as eptifibatide (Integrilinm) or tirofiban (Agrastat™).

The bioactive agent can be an immunosuppressive agent, for example, cyclosporine, CD-34 antibody, everolimus, mycophenolic acid, sirolimus, tacrolimus, and the like.

Other exemplary therapeutic antibodies include trastuzumab (Herceptin™), a humanized anti-HER2 monoclonal antibody (moAb); alemtuzumab (Campath™), a humanized anti-CD52 moAb; gemtuzumab (Mylotarg™), a humanized anti-CD33 moAb; rituximab (Rituxan™), a chimeric anti-CD20 moAb; ibritumomab (Zevalin™), a murine moAb conjugated to a beta-emitting radioisotope; tositumomab (Bexxar™), a murine anti-CD20 moAb; edrecolomab (Panorex™), a murine anti-epithelial cell adhesion molecule moAb; cetuximab (Erbitux™), a chimeric anti-EGFR moAb; bevacizumab (Avastin™), a humanized anti-VEGF moAb, ranibizumab (Lucentis™), an anti-vascular endothelial growth factor mAb fragment, satumomab (OncoScint™) an anti-pancarcinoma antigen (Tag-72) mAb, pertuzumab (Omnitarg™) an anti-HER2 mAb, and daclizumab (Zenapax™) an anti IL-2 receptor mAb.

Additionally, the bioactive agent can be a surface adhesion molecule or cell-cell adhesion molecule. Exemplary cell adhesion molecules or attachment proteins (such as extracellular matrix proteins including fibronectin, laminin, collagen, elastin, vitronectin, tenascin, fibrinogen, thrombospondin, osteopontin, von Willibrand Factor, bone sialoprotein (and active domains thereof), or a hydrophilic polymer such as hyaluronic acid, chitosan or methyl cellulose, and other proteins, carbohydrates, and fatty acids.

Exemplary cell-cell adhesion molecules include N-cadherin and P-cadherin and active domains thereof.

Exemplary growth factors include fibroblastic growth factors, epidermal growth factor, platelet-derived growth factors, transforming growth factors, vascular endothelial growth factor, bone morphogenic proteins and other bone growth factors, and neural growth factors.

Polynucleotides and derivatives thereof include natural and synthetically prepared DNA and RNA polymers, and chemical analogs thereof Polynucleotides also include oligonucleotides. Exemplary polynucleotides include antisense mRNA, morpholino oligos, siRNA, ribozymes, ssDNA and dsRNA.

Zones of bioactive agents can be formed in the body member in any suitable manner. A zone of bioactive agent can, in the least, be formed of the bioactive agent itself If a zone of bioactive agent is prepared in this manner, maximal loading of a bioactive agent in the inner space of the body member can be achieved. In these aspects the bioactive agent can be present in a matrix-free zone. For example, in some preparations, the bioactive agent is not present in a polymeric matrix of material within the body member. In some preparations, the bioactive agent can be present as a finely divided solid, powder, or any other appropriate physical form.

A zone of bioactive agent can be prepared by packing bioactive agent into a portion of the body member. The bioactive agent present in a composition may include optional additives if desired. For example, bioactive agent in dry or powder form can be placed into a portion of the body member and then compressed to form a mass of bioactive agent, which represents a zone within the body member. For example, a cylindrical shaped body member with apertures on the circumferential face of the body is placed on end on a flat surface. Bioactive agent is added in dry or semi-dry form in the body member. A rod with a diameter less or equal to the inner diameter of the body member is then used to compress the bioactive agent within the body member. The compression forces bioactive agent against the inner walls of the body member and stabilizes the mass of bioactive agent within the body member. The process can be repeated with a different bioactive agent to form two or more zones of bioactive agent within the inner space.

Optionally, a different process can be used to form the second zone of bioactive agent within the inner space. For example, a second zone can be formed by providing the bioactive agent in a polymeric matrix.

As another way of forming a first zone of bioactive agent, a composition is prepared by dissolving or suspending the bioactive agent in a suitable solvent, which is then used to form a first zone of bioactive agent. For example, a cylindrical shaped body member with apertures on the circumferential face of the body is placed on end on a flat surface. A first aperture set is temporarily sealed off so that the bioactive agent composition cannot flow through the apertures. For example, tape can be applied on the exterior surface of the body member to temporarily cover the first aperture set. The composition is added to the body member and then treated to form a solid mass including bioactive agent. The solid mass can be formed by one or more techniques, such as precipitation of the bioactive agent, drying of the composition, or evaporation of the solvent. The process can be repeated with a different dissolved or suspended bioactive agent to form two or more zones of bioactive agent within the inner space.

In some modes of preparation, the bioactive agent can be formed by dissolving or suspending the bioactive agent in a suitable solvent. Exemplary solvents include, but are not limited to, chloroform, water, alcohol, acetone, acetonitrile, ether, methyl ethyl ketone (MEK), ethyl acetate, tetrahydrofuran (THF), dioxane, methylene chloride, xylene, toluene, N,N-dimethylformamide (DMF), dimethyl sulfoxide (DMSO), N,N-dimethylacetamide (DMAC), N-methylpyrrolidone (NMP), combinations of these, and the like.

Optionally, the bioactive agent can include one or more additives, such as diluents, carriers, excipients, stabilizers, or the like. Some examples of materials which can serve as pharmaceutically acceptable carriers include, but are not limited to, sugars such as lactose, glucose and sucrose.

Buffers, acids, and bases can be present in combination with the bioactive agent to provide a desired pH. Agents to increase the diffusion distance of bioactive agents released from the body member can also be included.

The bioactive agent can also be present in the body member in combination with a preservative. The preservative can be an antioxidant. For example, the antioxidant can be a hydrophobic antioxidant. Exemplary hydrophobic antioxidants that can be employed include, but are not limited to, tocopherols (such as α-tocopherol, β-tocopherol, γ-tocopherol, δ-tocopherol, ε-tocopherol, zetap₁-tocopherol, zeta₂-tocopherol, and eta-tocopherol), and ascorbic acid 6-palmitate.

One or more hydrophilic antioxidant(s) can also be used. Examples of hydrophilic antioxidants include citric acid and sodium citrate.

Other preservatives include amino acids such as cysteine and lysine, and parabens, such as methyl or propyl paraben can be included with the bioactive agent.

The delivery device can also include an imaging material. The imaging material can be present in the body member, or, in preparations that include a polymeric matrix, can be present within the matrix of polymeric material along with bioactive agent. The imaging materials can facilitate medical imaging of the device once implanted. Medical imaging materials are well known. Exemplary imaging materials include paramagnetic material, such as nanoparticular iron oxide, Gd, or Mn, a radioisotope, and non-toxic radio-opaque markers (for example, cage barium sulfate and bismuth trioxide). Radiopacifiers (such as radio opaque materials) can be included in any fabrication method or absorbed into or sprayed onto the surface of part or all of the implant. The degree of radiopacity contrast can be altered by controlling the concentration of the radiopacifier within or on the implant. Common radio opaque materials include barium sulfate, bismuth subcarbonate, and zirconium dioxide. Other radio opaque materials include cadmium, tungsten, gold, tantalum, bismuth, platinum, iridium, and rhodium. In some embodiments, iodine can be employed for both its radiopacity and antimicrobial properties. This can be useful for detection of medical devices that are implanted in the body (that are emplaced at the treatment site) or that travel through a portion of the body (that is, during implantation of the device). Paramagnetic resonance imaging, ultrasonic imaging, x-ray means, fluoroscopy, or other suitable detection techniques can detect medical devices including these materials. In another example, microparticles that contain a vapor phase chemical can be used for ultrasonic imaging. Useful vapor phase chemicals include perfluorohydrocarbons, such as perfluoropentane and perfluorohexane, which are described in U.S. Pat. No. 5,558,854 (Issued 24 Sep. 1996); other vapor phase chemicals useful for ultrasonic imaging can be found in U.S. Pat. No. 6,261,537 (Issued 17 Jul. 2001).

In some aspects of the invention, bioactive agent is present in a polymeric matrix. The polymeric matrix can either be biostable (non-degradable within the body) or degradable. A composition can be prepared that includes a polymeric material with a selected amount of bioactive agent. The composition is then added to the inner space of the device to form a zone of a polymeric matrix with bioactive agent. Bioactive agent is released through the apertures from the polymeric matrix following implantation of the device within a subject.

A polymeric matrix with bioactive agent can be formed in the inner space of the device using a biostable polymer. Exemplary biostable polymers include, but are not limited to, polymers of acrylates, vinyl polymers (such as ethylene vinyl acetates), urethanes, ethylene-based polymers (such as ethylene terephthalates and ethylene oxide), and silicones. Biostable polymers can be permeable to the bioactive agent, which can be released by diffusion through the polymeric matrix, and out of the aperture set of the device.

In some cases poly(ethylene-co-vinyl acetate) is used to form the biostable matrix within the delivery device.

In some aspects, the device includes a polymeric matrix formed from a poly(alkyl(meth)acrylate) and/or a poly(aromatic(meth)acrylate), where “(meth)” will be understood by those skilled in the art to include such molecules in either the acrylic and/or methacrylic form (corresponding to the acrylates and/or methacrylates, respectively).

Examples of suitable poly(alkyl(meth)acrylates) include those with alkyl chain lengths from 2 to 8 carbons, inclusive, and with molecular weights from 50 kilodaltons to 900 kilodaltons. In one preferred embodiment the polymeric material includes a poly(alkyl (meth)acrylate) with a molecular weight of from about 100 kilodaltons to about 1000 kilodaltons, preferably from about 150 kilodaltons to about 500 kilodaltons, most preferably from about 200 kilodaltons to about 400 kilodaltons. An example of a particularly preferred polymer is poly (n-butyl methacrylate). Examples of other preferred polymers are poly(n-butyl methacrylate-co-methyl methacrylate, with a monomer ratio of 3:1, poly(n-butyl methacrylate-co-isobutyl methacrylate, with a monomer ratio of 1:1 and poly(t-butyl methacrylate). Such polymers are available commercially (e.g., from Sigma-Aldrich, Milwaukee, Wis.) with molecular weights ranging from about 150 kilodaltons to about 350 kilodaltons, and with varying inherent viscosities, solubilities and forms (e.g., as slabs, granules, beads, crystals or powder).

Examples of suitable poly(aromatic(meth)acrylates) include poly(aryl(meth)acrylates), poly(aralkyl(meth)acrylates), poly(alkaryl(meth)acrylates), poly(aryloxyalkyl(meth)acrylates), and poly (alkoxyaryl(meth)acrylates).

Examples of suitable poly(aryl(meth)acrylates) include poly(9-anthracenyl methacrylate), poly(chlorophenyl acrylate), poly(methacryloxy-2-hydroxybenzophenone), poly(methacryloxybenzotriazole), poly(naphthyl acrylate), poly(naphthylmethacrylate), poly-4-nitrophenylacrylate, poly(pentachloro(bromo, fluoro) acrylate) and methacrylate, poly(phenyl acrylate) and poly(phenyl methacrylate). Examples of suitable poly(aralkyl (meth)acrylates) include poly(benzyl acrylate), poly(benzyl methacrylate), poly(2-phenethyl acrylate), poly(2-phenethyl methacrylate) and poly(1-pyrenylmethyl methacrylate). Examples of suitable poly(alkaryl(meth)acrylates include poly(4-sec-butylphenyl methacrylate), poly(3-ethylphenyl acrylate), and poly(2-methyl-1-naphthyl methacrylate). Examples of suitable poly(aryloxyalkyl(meth)acrylates) include poly(phenoxyethyl acrylate), poly(phenoxyethyl methacrylate), and poly(polyethylene glycol phenyl ether acrylate) and poly(polyethylene glycol phenyl ether methacrylate) with varying polyethylene glycol molecular weights. Examples of suitable poly(alkoxyaryl(meth)acrylates) include poly(4-methoxyphenyl methacrylate), poly(2-ethoxyphenyl acrylate) and poly(2-methoxynaphthyl acrylate).

Acrylate or methacrylate monomers or polymers and/or their parent alcohols are commercially available from Sigma-Aldrich (Milwaukee, Wis.) or from Polysciences, Inc, (Warrington, Pa.).

The matrix can also be formed by a mixture of two or more biostable polymers. Exemplary mixtures of biostable polymers are described in U.S. Pat. No. 6,214,901 (Chudzik et al.) and U.S. Publication No. 2002/0188037 A1 (Chudzik et al.) (each commonly assigned to the assignee of the present invention). These documents describe polymer mixtures of poly(butylmethacrylate) (PBMA) and poly(ethylene-co-vinyl acetate) (pEVA).

Other useful mixtures of polymers that can be included in the coating are described in U.S. Publication No. 2004/0047911. This publication describes polymer blends that include poly(ethylene-co-methacrylate) and a polymer selected from the group consisting of a poly(vinyl alkylate), a poly(vinyl alkyl ether), a poly(vinyl acetal), a poly(alkyl and/or aryl methacrylate) or a poly(alkyl and/or aryl acrylate); not including pEVA.

The polymeric material can also be a styrene copolymer, such as poly(styrene-isobutylene-styrene); the preparation of medical devices having such coatings that include poly(styrene-isobutylene-styrene) is described in, for example, U.S. Pat. No. 6,669,980.

In other forms of the present invention, the body member includes a matrix comprising a biodegradable polymer. The matrix can be formed from a biodegradable polymer that degrades in aqueous environments, such as by simple hydrolysis. The matrix can be formed from a biodegradable polymer that is enzymatically degradable. For example, an enzymatically biodegradable polymer can be one that is degraded by enzymes produced by a mammalian body, or those that are produced by other lower organisms (such as bacteria). Once broken down, the degradation products of these polymers are typically gradually absorbed or eliminated by the body.

Examples of classes of synthetic polymers that have been studied as biodegradable materials include polyesters, polyamides, polyurethanes, polyorthoesters, polycaprolactone (PCL), polyiminocarbonates, aliphatic carbonates, polyphosphazenes, polyanhydrides, and copolymers thereof Specific examples of biodegradable materials that can be used in connection with the device of the invention include polylactide, polyglycolide, polydioxanone, poly(lactide-co-glycolide), poly(glycolide-co-polydioxanone), polyanhydrides, poly(glycolide-co-trimethylene carbonate), and poly(glycolide-co-caprolactone). Blends of these polymers with other biodegradable polymers can also be used. Typically, release of a bioactive agent occurs as these polymers dissolve or degrade in situ.

Biodegradable polyetherester copolymers can be used. Generally speaking, the polyetherester copolymers are amphiphilic block copolymers that include hydrophilic (for example, a polyalkylene glycol, such as polyethylene glycol) and hydrophobic blocks (for example, polyethylene terephthalate). Examples of block copolymers include poly(ethylene glycol)-based and poly(butylene terephthalate)-based blocks (PEG/PBT polymer). Examples of these types of multiblock copolymers are described in, for example, U.S. Pat. No. 5,980,948. PEG/PBT polymers are commercially available from Octoplus BV, under the trade designation PolyActive™.

Biodegradable copolymers having a biodegradable, segmented molecular architecture that includes at least two different ester linkages can also be used. The biodegradable polymers can be block copolymers (of the AB or ABA type) or segmented (also known as multiblock or random-block) copolymers of the (AB)ₙ type. These copolymers are formed in a two (or more) stage ring opening copolymerization using two (or more) cyclic ester monomers that form linkages in the copolymer with greatly different susceptibilities to transesterification. Examples of these polymers are described in, for example, in U.S. Pat. No. 5,252,701 (Jarrett et al., “Segmented Absorbable Copolymer”).

Other suitable biodegradable polymer materials include biodegradable terephthalate copolymers that include a phosphorus-containing linkage. Polymers having phosphoester linkages, called poly(phosphates), poly(phosphonates) and poly(phosphites), are known. See, for example, Penczek et al., Handbook of Polymer Synthesis, Chapter 17: “Phosphorus-Containing Polymers,” 1077-1132 (Hans R. Kricheldorf ed., 1992), as well as U.S. Pat. Nos. 6,153,212, 6,485,737, 6,322,797, 6,600,010, 6,419,709. Biodegradable terephthalate polyesters can also be used that include a phosphoester linkage that is a phosphite. Suitable terephthalate polyester-polyphosphite copolymers are described, for example, in U.S. Pat. No. 6,419,709 (Mao et al., “Biodegradable Terephthalate Polyester-Poly(Phosphite) Compositions, Articles, and Methods of Using the Same). Biodegradable terephthalate polyester can also be used that include a phosphoester linkage that is a phosphonate. Suitable terephthalate polyester-poly(phosphonate) copolymers are described, for example, in U.S. Pat. Nos. 6,485,737 and 6,153,212 (Mao et al., “Biodegradable Terephthalate Polyester-Poly(Phosphonate) Compositions, Articles and Methods of Using the Same). Biodegradable terephthalate polyesters can be used that include a phosphoester linkage that is a phosphate. Suitable terephthalate polyester-poly(phosphate) copolymers are described, for example, in U.S. Pat. Nos. 6,322,797 and 6,600,010 (Mao et al., “Biodegradable Terephthalate Polyester-Poly(Phosphate) Polymers, Compositions, Articles, and Methods for Making and Using the Same).

Biodegradable polyhydric alcohol esters can also be used (See U.S. Pat. No. 6,592,895). This patent describes biodegradable star-shaped polymers that are made by esterifying polyhydric alcohols to provide acyl moieties originating from aliphatic homopolymer or copolymer polyesters. The biodegradable polymer can be a three-dimensional crosslinked polymer network containing hydrophobic and hydrophilic components which forms a hydrogel with a crosslinked polymer structure, such as that described in U.S. Pat. No. 6,583,219. The hydrophobic component is a hydrophobic macromer with unsaturated group terminated ends, and the hydrophilic polymer is a dextran polysaccharide containing hydroxy groups that are reacted with unsaturated group introducing compounds. The components are convertible into a one-phase crosslinked polymer network structure by free radical polymerization.

The bioactive agent can also be delivered from a matrix comprising a poly(ester-amide) (PEA). Degradable poly(ester-amides) can include those formed from the monomers OH-x-OH, z, and COOH-y-COOH, wherein x is alkyl, y is alkyl, and z is an alpha-amino acid. Examples of such alpha-amino acids are glycine, alanine, valine, leucine, isoleucine, norleucine, cysteine, methionine, phenylalanine, tyrosine, and tryptophan. The device can be filled with a matrix including a blend of two or more PEAs and a bioactive agent. Exemplary PEAs and blends are described in U.S. Pat. No. 6,703,040 (Katsarava, et al.)

Another biodegradable material comprises α-1,4 glucopyranose polymers. Some exemplary α-1,4 glucopyranose polymers that can be used to form the matrix are low molecular weight starch-derived polymers as described in commonly assigned under U.S. Pub. No. 2005/0255142, published Nov. 17, 2005, (Chudzik et al.) and U.S. patent application Ser. No. 11/271,213, filed Nov. 11, 2005 (Chudzik et al.). These low molecular weight starch-derived polymers, as exemplified by amylose, maltodextrin, and polyalditol, comprise reactive groups, such as polymerizable groups, which can be activated to form a biodegradable matrix that includes bioactive agent.

The biodegradable polymer can comprise a polymer based upon α-amino acids (such as elastomeric copolyester amides or copolyester urethanes, as described in U.S. Pat. No. 6,503,538).

In some modes of practice, any one or more of the biodegradable polymers can be used to plug or cover the apertures of the device. The presence of a biodegradable polymers located in or on the aperture can delay or modulate release of bioactive agent from the device.

Various techniques can be performed to incorporate the bioactive agent in a polymeric matrix in the inner space of the device. For example, in some modes of preparation a liquid composition that includes the polymer and the bioactive agent is prepared and placed in a portion of the inner space of the body member. In order to prevent the composition from flowing out of the apertures, the apertures can be temporarily sealed using tape or a similar product. Other methods to prevent the composition from flowing out of the apertures may be used as well.

The liquid composition can then be treated to cause formation of a polymeric matrix that has a degree of solidity. For example, the composition can be treated to provide a semi-solid or solid polymeric matrix. Various types of treatments can be performed to provide the polymeric matrix depending on the properties of the polymers used and the nature of matrix formation.

For example, in some cases, the polymeric matrix is formed by removal of the solvent from the composition. For example, the composition can include a volatile organic solvent that can be removed by evaporation; upon evaporation the matrix is formed.

In other cases the matrix is formed by promoting a chemical reaction in the composition. The chemical reaction can be one that promotes the association of polymers in the composition through one or more bonds selected from ionic, covalent, coordinative, hydrogen and Van der Waals bonds.

In some preparations a cross-linking agent can be used to promote the association of polymers in a composition thereby forming a matrix. The choice of a particular crosslinking agent may depend on the ingredients of the composition including the polymer and bioactive agent.

Some exemplary crosslinking agents include two or more activatable groups, which can react with the polymers in the composition. Exemplary activatable groups include photoreactive group, which can be activated by UV light. The photoreactive group can be an aryl ketone, such as acetophenone, benzophenone, anthraquinone, anthrone, quinone, and anthrone-like heterocycles.

The photoactivatable cross-linking agent can be ionic, and can have good solubility in an aqueous composition. Thus, in some embodiments, at least one ionic photoactivatable cross-linking agent is used to form the coating. The ionic cross-linking agent can include an acidic group or salt thereof, such as selected from sulfonic acids, carboxylic acids, phosphonic acids, salts thereof, and the like. Exemplary counter ions include alkali, alkaline earths metals, ammonium, protonated amines, and the like.

Exemplary ionic photoactivatable cross-linking agents include 4,5-bis(4-benzoylphenylmethyleneoxy)benzene-1,3-disulfonic acid or salt; 2,5-bis(4-benzoylphenylmethyleneoxy)benzene-1,4-disulfonic acid or salt; 2,5-bis(4-benzoylmethyleneoxy)benzene-1-sulfonic acid or salt; N,N-bis[2-(4-benzoylbenzyloxy)ethyl]-2-aminoethanesulfonic acid or salt, and the like. See U.S. Pat. No. 6,278,018.

In some aspects a non-ionic photoactivatable cross-linking agent can be used. Exemplary non-ionic cross-linking agents are described, for example, in U.S. Pat. Nos. 5,414,075 and 5,637,460 (Swan et al., “Restrained Multifunctional Reagent for Surface Modification”). Chemically, the first and second photoreactive groups, and respective spacers, can be the same or different.

Some suitable cross-linking agents are those formed by a mixture of the chemical backbone molecule (such as pentaerythritol) and an excess of a derivative of the photoreactive group (such as 4-bromomethylbenzophenone). An exemplary product is the tetrakis (4-benzoylbenzyl ether) of pentaerythritol (tetrakis(4-benzoylphenylmethoxy-methyl)methane). See U.S. Pat. No. 5,414,075 (columns 7 and 8, lines 1-25 (Formula III) and U.S. Pat. No. 5,637,460.

If a cross-linking agent having latent reactive groups is included in the composition, a step of irradiating may be performed to activate the latent reactive group to promote formation of the matrix. Generally, the step of irradiating can be performed by subjecting the photoreactive groups to actinic radiation in an amount that promotes activation of the photoreactive group and formation of the matrix.

Actinic radiation can be provided by any suitable light source that promotes activation of the photoreactive groups. Preferred light sources (such as those available from Dymax Corp.) provide UV irradiation in the range of 190 nm to 360 nm. A suitable dose of radiation is in the range of from about 0.5 mW/cm² to about 2.0 mW/cm².

In some aspects, it may be desirable to use filters in connection with the step of activating the photoreactive groups. The use of filters can be beneficial from the standpoint that they can selectively minimize the amount of radiation of a particular wavelength or wavelengths that are provided to the composition during the activation process. This can be beneficial if the bioactive agent is sensitive to radiation of a particular wavelength(s), and may degrade or decompose upon exposure.

In some desired modes of practice, the matrix is formed using a polymer having pendent polymerizable groups, also known as “macromers.” A preferred polymerizable group is an ethylenically unsaturated group. Suitable ethylenically unsaturated groups include vinyl groups, acrylate groups, methacrylate groups, ethacrylate groups, 2-phenyl acrylate groups, acrylamide groups, methacrylamide groups, itaconate groups, and styrene groups.

Biostable or biodegradable macromers can be used to form the matrix. In some desired modes of practice, the matrix is formed using a biodegradable poly(α-1,4 glucopyranose) macromer described in U.S. Pub. No. 2005/0255142 and U.S. patent application Ser. No. 11/271,213, commonly assigned to the applicant. These low molecular weight starch-derived polymers, as exemplified by amylose, maltodextrin, and polyalditol, comprise reactive groups, such as polymerizable groups, which can be activated to form a biodegradable matrix that includes bioactive agent.

The polymer can be used at a concentration to provide a desired density of crosslinked natural biodegradable polysaccharide. Generally, higher concentrations of polymer will allow the formation of a denser matrix, which can reduce the rate of release of the polymer. In forms where biodegradable polymers are used to form the matrix, dense matrix can reduce the rate of degradation of the matrix, thereby reducing release of the bioactive agent.

In some embodiments the polymer in the composition has a concentration in the range of 5-100% (w/v), and 5-50%, and in more specific embodiments in the range of 10-20% and in other embodiments in the range of 20-50% (w/v).

Matrix formation can be caused by polymerization of the macromers in the composition using a suitable initiator system. As used herein, an “initiator” refers to a compound, or more than one compound, that is capable of promoting the formation of a reactive species from the coupling group. For example, the initiator can promote a free radical reaction of a polymerizable group on a macromer. In some modes of practice, polymerization is carried out using a photoinitiator. Suitable photoinitiators, which can also be utilized as cross-linking agents having photoreactive groups, are described herein. An amount of photoinitiator in the range of about 0.1% (w/v) to about 10% (w/v), or in the range of about 1% (w/v) to about 5% (w/v), can be present in the composition.

As one way of forming a polymeric matrix including bioactive agent within the body member, a composition including macromer, bioactive agent, and a photoinitiator is placed within a portion of the body member. In order to prevent the composition from flowing out of the apertures, the apertures can be temporarily sealed using tape or a similar product. Desirably, a tape is used that allows light (such as UV light which activates the photoinitiator) to be passed through the apertures in order to irradiate the composition in the body member to promote matrix formation. The composition that is disposed within the body member is then treated with UV irradiation to promote matrix formation. The process can be repeated using a different composition to form another polymeric matrix containing bioactive agent within the body member.

In other cases, light capable of activating the photoinitiator is passed through the body member and into composition. Such a method of matrix formation can be used with substrate materials that are transparent.

In some aspects, the initiator includes an oxidant/reductant pair, a “redox pair,” to drive polymerization of the biodegradable polysaccharide. In this case, polymerization of the biodegradable polysaccharide is carried out upon combining one or more oxidants (e.g., a first member of the redox pair) with one or more reductants (e.g., a second member of the redox pair). Other compounds can be included in the composition to promote polymerization of the biodegradable polysaccharides.

The oxidizing agent can be selected from inorganic or organic oxidizing agents, including enzymes; the reducing agent can be selected from inorganic or organic reducing agents, including enzymes. Exemplary oxidizing agents include peroxides, including hydrogen peroxide, metal oxides, and oxidases, including glucose oxidase. Exemplary reducing agents include salts and derivatives of electropositive elemental metals such as Li, Na, Mg, Fe, Zn, Al, and reductases. In one mode of practice, the reducing agent is present at a concentration of about 2.5 mM or greater when the reducing agent is mixed with the oxidizing agent. Prior to mixing, the reducing agent can be present in a composition at a concentration of, for example, 5 mM or greater.

In order to provide a matrix using redox chemistry, a composition including macromer and bioactive agent is added to the body member bioactive agent, and a redox reaction is allowed to take place within the body member, thereby forming the matrix. This can be accomplished in a number of different ways. In one mode, the first and second members of the redox pair are combined in the presence of macromer and bioactive agent, and the mixture is immediately added to the body member. In another mode, the first and second members of the redox pair are combined in the presence of macromer and bioactive agent within the body member. This can be accomplished, for example, by mixing a composition that includes the macromer, bioactive agent, and an oxidizing agent with a composition that includes a macromer, bioactive agent, and reducing agent, in the body member. As another example, a composition that includes the macromer, bioactive agent, and a member of the redox pair is added to the body member that includes the other member of the redox pair. For example, the composition can include an oxidizing agent, and the body member can include a reducing agent. For example, the reducing agent can be disposed on the inner walls of the body member by dip coating or injecting the body member with a solution of bioactive agent also containing the oxidant.

As another example, the matrix can be formed by ionically crosslinking polymers together in the composition. One exemplary method involves crosslinking of alginate polymers using calcium ions. Alginate crosslinking is known in the art and has been used extensively for encapsulating cellular material (see, for example, U.S. Pat. No. 4,407,957).

The matrix of material within the body member can be formed before and/or after the device is placed at a target location within a subject. While forming a polymeric matrix before the device is implanted within a subject can facilitate the preparation of the device, there can be advantages to forming the polymeric matrix following a step of implanting the device.

For example, the body member of the device without all or a portion of the bioactive agent within may be delivered to a target site within the body. A matrix-forming composition can be delivered to the device in situ using a delivery instrument such as a catheter or microcatheter. Matrix formation can be promoted after a portion of the body member is filled with the composition.

The body member can also be refilled with bioactive agent. For example, some devices can be implanted at a target location and for a period of time in which bioactive agent is depleted from the inner space of the body member. After all or a portion of the bioactive agent is depleted, the inner space can be refilled with bioactive agent in situ. In some cases, the process of refilling can be carried out using a matrix-forming composition as described herein.

Upon implantation in a subject, the device can release the bioactive agents through their respective aperture sets at a desired timing and rate. The bioactive agents can be present in the body member so that they are released through the apertures in any suitable manner. Generally, upon implantation of the body member into a target area of a subject, body fluids promote the release of the bioactive through the apertures of the body member.

Various release mechanisms are contemplated. One type of release mechanism involves the release of the bioactive agent through the apertures when the action of the body fluid dissolves the bioactive agent in the inner space. In this mechanism, it is not required that the bioactive agent is immobilized in a polymeric matrix.

Other types of release mechanisms involve release of the bioactive agent from a polymeric matrix. Release mechanisms from various biostable and biodegradable polymeric matrices can be used.

One type of release mechanism involves the release of the bioactive agent through the apertures from a hydrophobic biostable polymeric matrix. Generally, bioactive agent is released from the device through the apertures by diffusion of the bioactive agent out of the matrix. The rate of release of bioactive agent can be slowed by the overall configuration of the body member. The rate of release can be modulated by the total aperture area, with the release rates increasing as the aperture area increases.

After the device has been implanted in a patient, bioactive agent is released from the hydrophobic matrix through the apertures. A gradient of bioactive agent within the matrix is established, with higher concentrations of bioactive agent found in areas of the matrix that are distal from the aperture(s), and lower concentrations of bioactive agent found near the apertures. In combination with the apertures, the hydrophobic matrix can be prepared to regulate the release of the drugs while using a matrix which has a structure which promotes the total release of the bioactive agents.

Another type of release mechanism involves the release of the bioactive agent through the apertures from a hydrophilic biostable polymeric matrix. The device of the present invention can advantageously control swelling of the hydrophilic biostable polymeric matrices, which otherwise swell rapidly and lose bioactive agent due to water being driven into the matrix.

The present invention provides a device configuration that allows bioactive agents present in the hydrophilic matrix to be released in a controlled manner. The apertures can be used to limit the influx of water, which in turn limits increases in osmotic pressure. The body member can also limit the swelling of the matrix, which controls the matrix properties and release of the bioactive agent.

Another type of release mechanism involves the release of the bioactive agent through the apertures from a degradable polymeric matrix. One type of polymeric matrix is formed from a bulk erodable polymer, such as poly(lactide), poly(glycolide), and poly(caprolactone). The present device can improve the release of bioactive agents from matrices formed from these types of polymers.

The device of the present invention can provide for controlled release of bioactive agents contained within a bulk erodable polymeric matrix. In some regards, the device can limit the amount of bioactive agent released from the matrix. The device can also be used to maintain the integrity of the matrix. For example, bulk erosion of the matrix may cause portions of the matrix to break down into particulates. The body member can prevent these particulates from being dislodged from the rest of the matrix, which may result in the rapid release of bioactive agent.

Release of the bioactive agent can also be from a polymeric matrix formed from an enzymatically degradable polymer. Exemplary biodegradable polymers include natural biodegradable polysaccharides such as amylose, maltodextrin, and polyalditol as described herein. In some aspects, the matrix degrades by surface erosion. Body fluids including enzymes, e.g. amylase, capable of degrading the matrix enter the apertures and contact the matrix. Upon surface contact, the enzyme erodes the surface of the matrix, resulting in release of the bioactive agent from that area of the matrix.

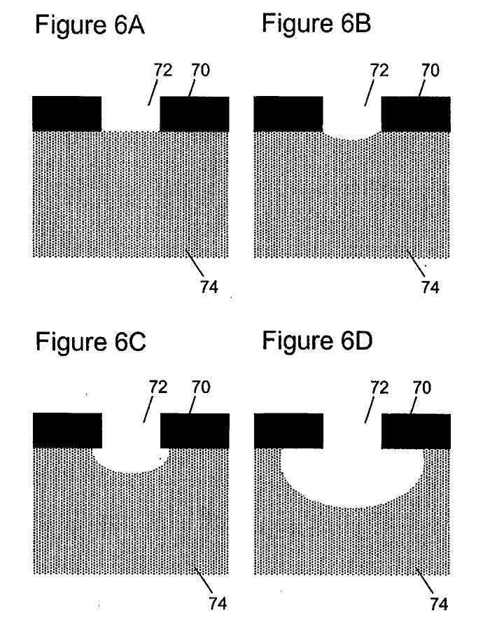

An illustration of a time course of degradation of the surface erodable matrix in the present invention is exemplified by FIG. 6a-6A. FIG. 6a shows a cross section of a portion of the device with the body member 70, aperture 72 and, biodegradable matrix 74 (having bioactive agent) prior to contact with body fluid having enzyme capable of degrading the matrix. In FIG. 6B the device is implanted for a first period of time and degradation of a portion of the matrix occurs. In FIG. 6C the device is implanted for a second period of time and degradation of greater portion of the matrix occurs. In FIG. 6D the device is implanted for a third period of time and degradation of greater portion of the matrix occurs.

### EXAMPLE 1

Two drug delivery device cylinders are joined together to form the body member of a delivery device. The two hollow stainless steel metal tubes (Small Parts, Inc. Logansport, Ind.), tube A and tube B, are obtained, and each tube has a diameter of approximately 1.00 mm and a length of 10.00 mm. One end of each tube (the distal end) is sealed. Tube A is filled with a matrix-forming composition containing maltodextrin-acrylate and bioactive agent, FITC-Dextran, as described in Example 19 of U.S. Patent Publication 2005/0255142 and a biodegradable matrix is formed. Tube B is filled with a matrix of poly(ethylene-co-vinylacetate) and poly(n-butyl methacrylate) and bioactive agent, β-estradiol, as described in Example 3 of U.S. patent publication 2003/0232087. The implantable drug delivery device, which has Tubes A and B joined together at the distal ends of each tube to make Tube AB, is complete. Apertures are functional at both the distal and proximal ends of Tube AB.

Tube AB is evaluated for controlled release of the two bioactive agents, β-estradiol and FITC-Dextran. The construction of Tube AB shows how to protect dissimilar bioactive agents and achieve controlled release of each of the bioactive agents.

As described in this Example, Tube AB is a stainless steel tube comprising a biodegradable polymeric matrix and bioactive agent in the inner space of Tube A, and a durable polymeric matrix and a different bioactive agent in the inner space of Tube B. In other embodiments, at least a portion of the tube, or other geometry, could be biodegradable.

Many different configurations are possible for the polymeric matrix placed within the inner space. While this example describes the combination of a biodegradable and biostable matrix, both matrices could be biodegradable, biostable, or a mixture of biostable and biodegradable polymer compositions to make a hybrid matrix. Additionally, the bioactive agents could be different or the same. If the bioactive agents are the same in the two bioactive containing matrices, one of the bioactive agents could be at a higher concentration than the other bioactive agent. For further controlled release of the bioactive agents, additional apertures of different sizes and incidence could be made in the walls of the tube.

### EXAMPLE 2

In this example, one cylinder with two bioactive agents within the inner space forms the drug delivery device. A stainless steel tube (Small Parts, Inc. Logansport, Ind.), Tube C, is obtained and has a diameter of approximately 1.00 mm and a length of 10.00 mm. The distal end is temporarily sealed. Approximately 50% of Tube C is filled with a matrix-forming composition containing maltodextrin-acrylate and bioactive agent, FITC-Dextran, as described in Example 19 of U.S. patent publication 2005/0255142. After crosslinking the maltodextrin-acrylate to form matrix in the first portion of the tube, the rest of Tube C is filled with a matrix-forming composition containing maltodextrin-acrylate and a different bioactive agent. The bioactive agent in the matrix of the second portion of Tube C is a protein, for example IgG, which is prepared in a manner to make the protein not only compatible with the crosslinkable matrix but also elutable from the polymeric matrix and Tube C in a controllable manner. The temporary seal is removed from the distal end of Tube C leaving apertures at both the distal and proximal ends. Tube C is evaluated for controlled release of the two bioactive agents, FITC-Dextran and IgG.

Sequentially filling of Tube C with two dissimilar bioactive agents in a polymeric matrix to achieve controlled release of each of the bioactive agents is one method to prepare a drug delivery device. Another method is the simultaneous injection of the bioactive containing matrices from both ends of the tube. With either method, the construction by simultaneous or sequential injection of bioactive containing polymeric matrices into Tube C makes an implantable drug delivery device that protects dissimilar bioactive agents and achieves controlled release of each of the bioactive agents.

As in Example 1, Tube C has many possible configurations. While this example describes the combination of a biodegradable and biostable polymeric matrix, both matrices could be biodegradable, biostable, or a mixture of biostable and biodegradable polymer compositions to make a hybrid matrix. Crosslinking or curing the first polymeric matrix containing bioactive agent in the first portion of the tube prior to disposing the second polymeric matrix containing bioactive agent in the second portion of the tube is one way of preventing substantial mixing of the multiple polymeric matrices containing bioactive agents. Optionally, the second polymeric matrix may be cured or crosslinked within the inner space to hold the matrix in place within the second portion of the tube. Such crosslinking procedures are one method available to prevent the mixing of two dissimilar bioactive containing polymeric matrices. Other methods may be used depending on whether a sequential or simultaneous loading process is used for loading the bioactive agent containing polymeric matrices into a tube. For example, inserting a physical barrier into the tube to prevent mixing of the compositions prior to injecting the bioactive containing polymeric matrices is one such method. Additionally, the bioactive agents could be different or the same. If the bioactive agents are the same in the two bioactive containing matrices, one of the bioactive agents could be at a higher concentration than the other bioactive agent. For further controlled release of the bioactive agents, additional apertures of different sizes and incidence could be made in the walls of the tube. In other embodiments, at least a portion of the tube, or other geometry, could be biodegradable.

What is claimed is:

1. An implantable device for the delivery of at least two bioactive agents to a subject, the device comprising:
a body member comprising
a first set of apertures,
a second set of apertures, and
an inner space comprising
an amount of a first bioactive agent, and
an amount of a second bioactive agent,
wherein, during implantation in a subject, the majority of the amount of the first bioactive agent is releasable from the device through the first set of apertures, and the majority of the amount of the second bioactive agent is releasable from the device through the second set of apertures.

2. The device of claim 1 wherein the body member comprises a biodegradable polysaccharide.

3. The device of claim 1 wherein the first and second sets comprise one or more apertures.

4. The device of claim 1 wherein an aperture has an area of 12.5 mm² or less on average.

5. The device of claim 1 wherein an aperture has an area in the range of 3×10³ μm² to 3.25×10⁵ μm².

6. The device of claim 1 wherein the apertures comprise a material that is dissolvable or degradable during implantation in a subject.

7. The device of claim 1 comprising a total aperture area and a total body member surface area, and the ratio of the total aperture area to the total body member area is 2:3 or less.

8. The device of claim 7 wherein the ratio of the total aperture area to the total body member area is in the range of 1:10 to 1:207.

9. The device of claim 1 wherein the first set of apertures has a total aperture area that is less than a total aperture area of a second set of apertures.

10. The device of claim 1 wherein the inner space comprises at least one polymeric matrix, and the first bioactive agent and second bioactive agent are present in the at least one polymeric matrix.

11. The device of claim 10 wherein the at least one polymeric matrix comprises a biodegradable polysaccharide.

12. The device of claim 1 wherein the first bioactive agent is hydrophilic.

13. The device of claim 1 wherein the second bioactive agent is hydrophobic.

14. The device of claim 1 wherein the first bioactive agent comprises a polypeptide.

15. The device of claim 1 wherein the second bioactive agent has a molecular weight of less than 1000 Da.

16. The device of claim 1 wherein the first bioactive agent is water-soluble and the second bioactive agent is poorly soluble, or not soluble in water.

17. A method for forming an implantable bioactive agent delivery device comprising steps of:
obtaining a body member comprising
a first set of apertures,
a second set of apertures, and
an inner space;
providing a first bioactive agent to a portion of the inner space so the first bioactive agent is primarily releasable through the first set of apertures during implantation of the device in a subject; and
providing a second bioactive agent to a portion of the inner space so the second bioactive agent is primarily releasable through the second set of apertures during implantation of the device in a subject.

18. A method for delivering two or more bioactive agents to a subject comprising steps of
implanting at a target location in the body an implantable bioactive agent delivery device comprising:
a body member comprising
a first set of apertures, and
a second set of apertures; and
an inner space comprising
a first bioactive agent, and
a second bioactive agent; wherein the first bioactive agent and the second bioactive agent are substantially unmixed in the inner space, and
allowing release of the first bioactive agent through the first set of apertures and release of the second bioactive agent through the second set of apertures in the body.

19. The method of claim 18 wherein the step of allowing release, the first bioactive agent has a rate of release that is less than the rate of release of the second bioactive agent

20. The method of claim 19 wherein the step of allowing release, the rate of release of the first bioactive agent is modulated by the first set of apertures having a total aperture area less than a total aperture area of a second set of apertures.

21. The method of claim 18 wherein the step of implanting, the first bioactive agent is present in a first polymeric matrix and the second bioactive agent is present in a second polymeric matrix.

22. The method of claim 21 wherein the step of allowing release, the rate of release of the first bioactive agent is modulated by a first polymeric matrix having a degree of crosslinking that is higher than the second polymeric matrix.

23. The method of claim 18, wherein the step of implanting comprises implanting the device into a portion of the eye.

24. An implantable device for the delivery of at least two bioactive agents to a subject, the device comprising:
a body member comprising
a first set of apertures,
a second set of apertures, and
an inner space comprising
a first bioactive agent and
a second bioactive agent, wherein the first bioactive agent and the second bioactive agent are substantially unmixed in the inner space, and
wherein, during implantation in a subject, the first bioactive agent is releasable from the device through the first set of apertures, and the second bioactive agent is releasable from the device through the second set of apertures.
